(19) United States
(12) Patent Application Publication
(10) Pub. No.: US 2009/0209871 A1
(43) Pub. Date: Aug. 20, 2009

(54) APPARATUS FOR MEASURING BLOOD FLOW RATE AND METHOD FOR MEASURING BLOOD FLOW RATE

(75) Inventors: Nobuaki Ueki, Kanagawa (JP); Ryoji Ishii, Kanagawa (JP)

Correspondence Address: FILDES & OUTLAND, P.C., 20916 MACK AVENUE, SUITE 2, GROSSE POINTE WOODS, MI, 48236, US

(73) Assignee: FUJI XEROX CO., LTD., Tokyo (JP)

(21) Appl. No.: 12/196,412

(22) Filed: Aug. 22, 2008

(30) Foreign Application Priority Data
Feb. 15, 2008 (JP) 2008-034957

(51) Int. Cl. A61B 5/02; A61B 5/00

(52) U.S. Cl. 600/504; 600/310

(57) ABSTRACT

Provided is an apparatus for measuring blood flow rate that includes a light emitting portion for irradiating living tissues with laser light, a photo-detector for detecting at least one of reflection, scattering, or absorption of the laser light, and an operation portion for calculating blood flow rate based on the difference between the spectrum of the laser light from the light emitting portion and the spectrum of the light detected by the photo-detector. The spectrum of the laser light has plural peaks.

### CROSS-REFERENCE TO RELATED APPLICATION

This application is based on and claims priority under 35 USC 119 from Japanese Patent Application No. 2008-034957 filed Feb. 15, 2008.

### BACKGROUND

1. Technical Field

This invention relates to an apparatus for measuring blood flow rate, and a method for measuring blood flow rate.

2. Related Art

Blood flow rate sensors that measure the velocity of the blood flowing in blood vessels in living tissues may be roughly classified into two types; optical type and ultrasonic type. The optical type, especially a laser type that uses the laser Doppler effect, provides higher resolution and can measure in a non-invasive way the flow rate of the blood in capillaries of peripheral tissues that is hard to measure by using the ultrasonic type.

An aim of the present invention is to provide an apparatus for measuring blood flow rate with higher accuracy than a case using a single mode oscillation laser.

### SUMMARY

An aspect of the present invention provides an apparatus for measuring blood flow rate that includes a light emitting portion for irradiating living tissues with laser light, a photo-detector portion for detecting at least one of reflection, scattering, or absorption of the laser light, and an operation portion for calculating blood flow rate based on a difference between the spectrum of the laser light from the light emitting portion and the spectrum of the light detected by the photo-detector portion. The spectrum of the laser light has plural peaks.

### BRIEF DESCRIPTION OF THE DRAWINGS

Exemplary embodiments of the present invention will be described in detail based on the following figures, wherein:

FIG. 1 is a schematic diagram for illustrating a method for measuring blood flow rate using an apparatus for measuring blood flow rate;

FIG. 2 is a diagram illustrating the principle of an apparatus for measuring blood flow rate of the present invention;

FIG. 3 is a block diagram illustrating a configuration of an apparatus for measuring blood flow rate according to an example of the present invention;

FIG. 4 is a block diagram illustrating a configuration of a photo-detector portion and an operation portion;

FIG. 5 is a diagram illustrating a method for calculating blood flow rate;

FIG. 6A illustrates a plan view of a configuration of a traverse multi-mode VCSEL device used for a light emitting portion of an example;

FIG. 6B is a cross sectional view of FIG. 6A taken along line A-A;

FIG. 7 is a flowchart illustrating a method for measuring blood flow rate using an apparatus for measuring blood flow rate according to an example;

FIG. 8 illustrates a specific configuration of an apparatus for measuring blood flow rate; and

FIGS. 9A and 9B illustrate examples of a configuration of an optical module.

### DETAILED DESCRIPTION

Referring to the accompanying drawings, exemplary embodiments for implementing an apparatus for measuring blood flow rate according to an aspect of the present invention will be now described. In an illustrated example, living tissues in a human body are irradiated with laser light in order to measure blood flow rate in the living tissues.

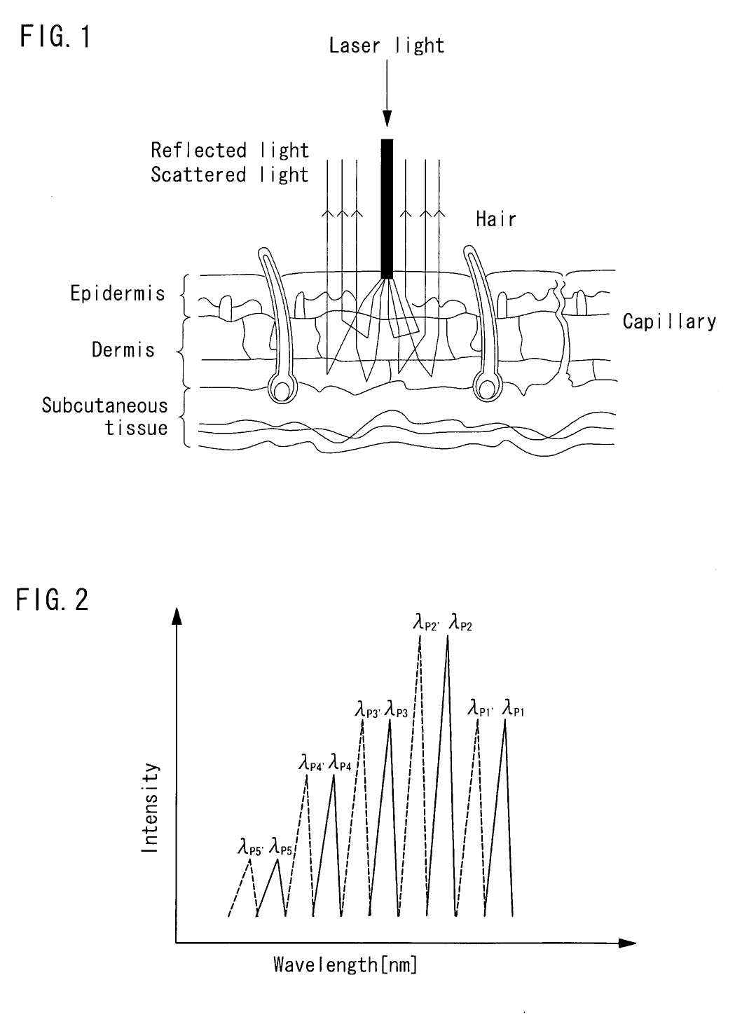

FIG. 1 is a schematic diagram for illustrating a method for measuring blood flow rate using an apparatus (sensor) for measuring blood flow rate. As shown in FIG. 1, human skin has three layers; the epidermis, the dermis, and the subcutaneous tissue. The epidermis protects tissues underneath the skin such as muscles, nerves, and blood vessels from injury. The dermis underneath the epidermis is a thick layer made up of fibrous tissue and elastic tissue, and the dermis contains nerve endings, sebaceous glands, sweat glands, and blood vessels. The blood vessels (capillaries, peripheral blood vessels or the like) in the dermis supply nutrients to the skin and regulate body temperatures. The number of the blood vessels varies depending on the portion of the body.

When the skin is irradiated with laser light having a specific wavelength, the laser light may penetrate through the epidermis, and the light may be reflected or scattered by the blood vessels in the dermis or by blood cells flowing in the blood vessels. Among the reflected light or scattered light, the light reflected or scattered by the blood cells flowing in the blood vessels experiences wavelength shift or frequency shift caused by the Doppler effect. From the amount of the shift due to the Doppler effect, blood flow rate can be calculated.

Referring now to FIG. 2, the principle of measuring blood flow rate according to the present invention will be described. In the present invention, laser light whose light spectrum has plural peaks is used as a light source for measurement. In FIG. 2, as an example, laser light having five peak wavelengths (λP1, λP2, λP3, λP4, and λP5, shown in solid lines) is shown. As long as the temperature is kept constant, these oscillation peak wavelengths have variations as small as fluctuations and wavelength intervals do not vary.

If living tissues are irradiated with such laser light, when the laser light is reflected by flowing blood, the wavelength of the laser light is shifted by the laser Doppler effect. The peak wavelengths of the laser light whose wavelengths are shifted may be expressed as λP1′, λP2′, λP3′, λP4′, and λP5′, shown in broken lines in FIG. 2. The amount of the wavelength shift, i.e., difference, for each peak can be expressed as Δλ1=|λP1−λP1′|, Δλ2=|λP2−λP2′|, Δλ3=|λP3−λP3′|, Δλ4=|λP4−λP4′|, and Δλ5=|λP5−λP5′|. By obtaining the arithmetic average of the amount of wavelength shift for each of these peak wavelengths, the amount of variation by the Doppler effect can be determined. The blood flow rate can be calculated from the determined amount of variation by the Doppler effect.

As described above, the laser light having plural peaks is used for a light source of the measurement and the amount of variation by the Doppler effect is determined from the amount of wavelength shift for each of the plural peaks, and thus the accuracy of the measurement becomes higher and the blood flow rate can be measured more accurately than a case where the amount of variation by the Doppler effect is determined from the shift of the wavelength having a single peak. In addition, the use of the laser light having plural peaks for a light source has a same effect as the use of plural laser light beams having plural wavelengths for a light source. Therefore, the use of the light source as in the present invention can make the light source in a measuring apparatus smaller and reduce the cost of the measuring apparatus. In the example, the amount of variation by the Doppler effect is determined from the amount of wavelength shift for each peak of the laser light; however, the amount of variation by the Doppler effect may be determined from the amount of frequency shift for each peak.

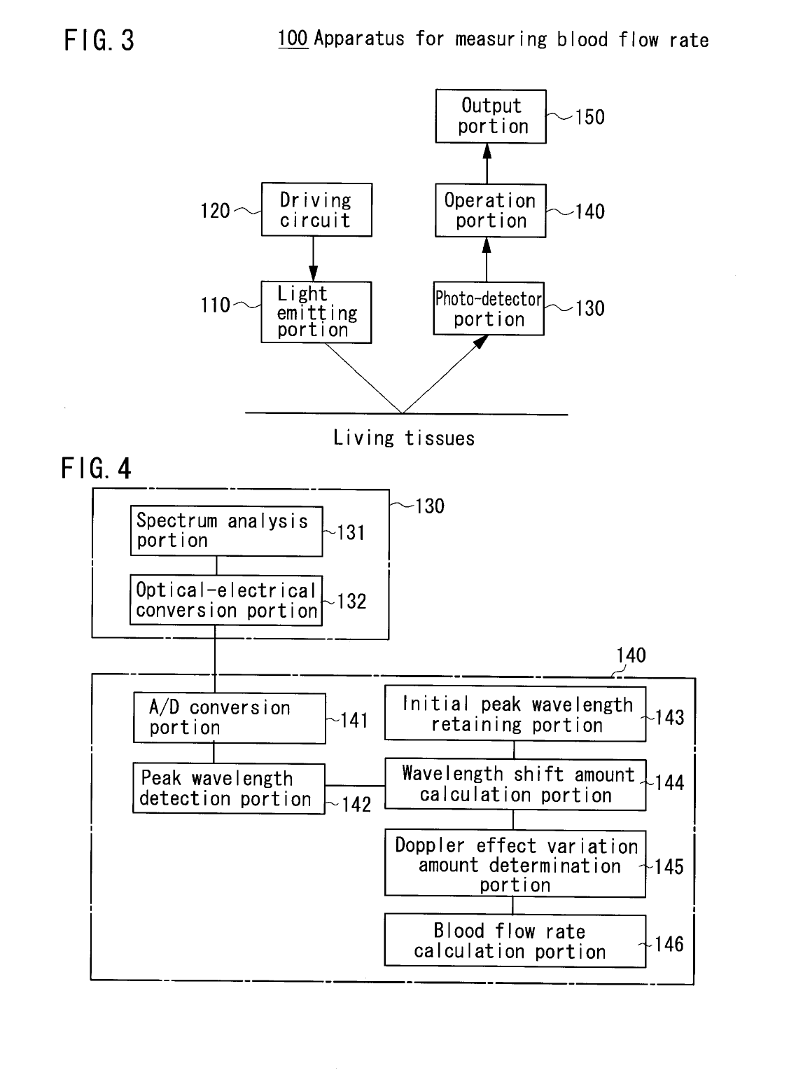

An apparatus for measuring blood flow rate according to an example will be described in detail. FIG. 3 is a block diagram of an apparatus for measuring blood flow rate. An apparatus for measuring blood flow rate 100 in an example may include a light emitting portion 110, whose light source is laser light having plural peak wavelengths as described above, for irradiating living tissues with the laser light, a driving circuit 120 for driving the light emitting portion 110, a photo-detector portion 130 for receiving light reflected or scattered from the living tissues using a light receiving device such as a photo diode or the like, and converting the light into an electrical signal, an operation portion 140 for receiving a detection signal outputted from the photo-detector portion 130 and calculating blood flow rate based on the detection signal, and an output portion 150 for displaying calculated blood flow rate on a display or the like.

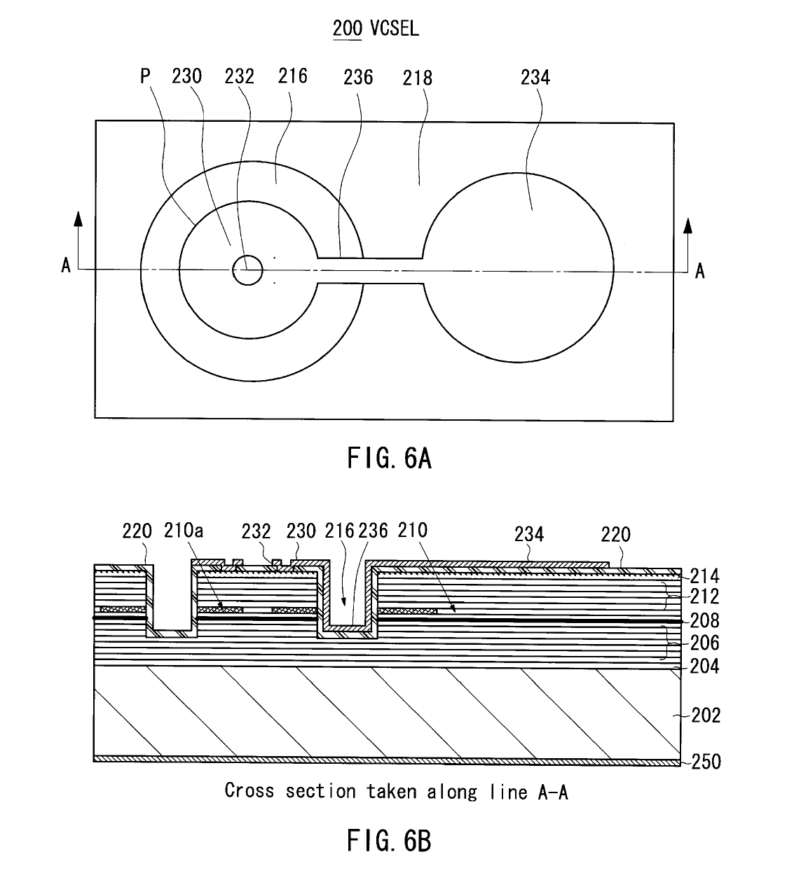

The light emitting portion 110 of this example preferably uses a traverse multi-mode VCSEL (Vertical-Cavity Surface-Emitting Laser diode, hereinafter referred to as VCSEL) device. The traverse mode exited by a multi-mode VCSEL device shows plural vertical modes, as shown in FIG. 2, each having a corresponding different peak wavelength. FIG. 6A is a plan view of a traverse multi-mode VCSEL device, and FIG. 6B is a cross sectional view of FIG. 6A taken along line A-A.

As shown in FIG. 6A and FIG. 6B, a VCSEL 200 includes an n-side electrode 250 on a back surface of an n-type GaAs substrate 202. Stacked on the substrate 202 are semiconductor layers including; an n-type GaAs buffer layer 204, a lower Distributed Bragg Reflector (DBR) 206 made of n-type AlGaAs semiconductor multilayer films, an active region 208, a current confining layer 210 made of p-type AlAs, an upper DBR 212 made of p-type AlGaAs semiconductor multilayer films, and a p-type GaAs contact layer 214.

In the substrate 202, a ring shaped groove 216 is formed by etching the semiconductor layers such that the groove 216 has a depth from the contact layer 214 to a portion of the lower DBR 206. By the groove 216, a cylindrical post P that becomes a laser light emitting portion is defined, and a pad formation region 218 is formed isolated from the post P. In the post P, a resonator structure is formed by the lower DBR 206 and the upper DBR 212, and therebetween, the active region 208 and the current confining layer 210 are interposed. The current confining layer 210 includes an oxidized region 210a in which AlAs being exposed on the side surface of the post P is selectively oxidized, and a conductive region surrounded by the oxidized region. The current confining layer 210 may confine current and light in the conductive region. The shape of the conductive region in a plan view is a round shape that reflects the outline of the post P.

On the entire surface of the substrate including the groove 216, an interlayer insulating film 220 is formed. At a top portion of the post P an annular contact hole is formed in the interlayer insulating film 220. Through the contact hole, a p-side round-shaped upper electrode 230 is electrically connected to the contact layer 214. At a center portion of the upper electrode 230, a round-shaped opening 232 that defines a laser light emitting portion is formed. In the pad formation region 218, a round-shaped electrode pad 234 is formed through the interlayer insulating film 220. The electrode pad 234 is connected to the p-side upper electrode 230 via an extraction electrode wiring 236 that extends in the groove 216.

In a VCSEL having such a configuration, in order to emit traverse multi-mode laser light, the outer diameter of the conductive region of the current confining layer 210 is preferably at least equal to or greater than 5 micrometers, and more preferably equal to or greater than 8 micrometers. If the outer diameter of the conductive region is smaller than 5 micrometers, the laser light becomes single-mode. The number of peak wavelengths varies in proportion to the size of the outer diameter of the conductive region. Therefore, the outer diameter of the conductive region can be selected in accordance with a desired number of the peaks.

The driving circuit 120 shown in FIG. 3 may apply a forward bias current to the n-side electrode 250 and the p-side electrode 230 of the VCSEL 200. This enables the VCSEL 200 to emit laser light having plural oscillation peaks at around 850 nm from the opening 232 approximately perpendicularly to the substrate.

FIG. 4 illustrates an example of a configuration of the photo-detector portion 130 and the operation portion 140. The photo-detector portion 130 may include a spectrum analysis portion 131 for analyzing the spectrum of the light reflected or scattered from the living tissues, and an optical-electrical conversion portion 132 for converting the light whose spectrum is analized into an electrical signal.

The operation portion 140 may include an A/D conversion portion 141 for converting an analog detection signal outputted from the optical-electrical conversion portion 132 into a digital signal, a peak wavelength detection portion 142 for detecting a peak wavelength that is shifted by the Doppler effect in the reflected light or scattered light from the converted digital signal, an initial peak wavelength retaining portion 143 for retaining the peak wavelength of the laser light (laser light having no variation by the Doppler effect) emitted by the light emitting portion 110, a wavelength shift amount calculation portion 144 for calculating the difference between the wavelength for each peak detected by the peak wavelength detection portion 142 and the wavelength for each peak retained by the initial peak wavelength retaining portion 143, that is, the amount of wavelength shift Δλ, a Doppler effect variation amount determination portion 145 for calculating, for example, the average of the amount of wavelength shift Δλ for each peak and determining the amount of variation by the Doppler effect, and a blood flow rate calculation portion 146 for calculating blood flow rate based on the amount of variation by the Doppler effect.

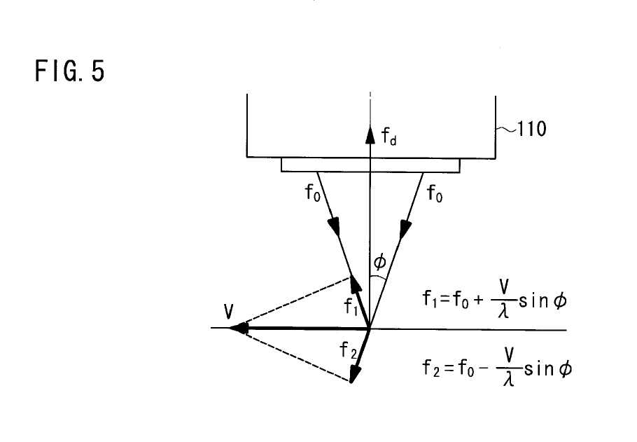

Referring now to FIG. 5, a preferable method for calculating blood flow rate in a blood flow rate calculation portion will be described. The laser light emitted from the light emitting portion (VCSEL) 110 may be split in two beams by a beam splitter, and an object to be measured may be irradiated with the split light beams at a crossing angle φ. When the speed of a moving object to be measured is V, the laser wavelength is λ, the frequency of the split laser light beams is f0, and the frequencies of the scattered light beams in which variation is caused by the Doppler effect are f1 and f2, then the differential frequency fd can be expressed by following equation (1).

$${f}_{d} = \vert {f}_{1} - {f}_{2} \vert = ( {f}_{0} + \frac{V}{λ} sin φ ) - ( {f}_{0} - \frac{V}{λ} sin φ ) \qquad ( 1 )$$

Where, φ is the crossing angle of the irradiated light, and λ is the laser wavelength. If the shift from a direction perpendicular to the object to be measured is taken into consideration, the differential frequency fd can be expressed by following equation (2).

$${f}_{d} = \frac{2 V}{λ} sin φ \cdot cos Δφ \qquad ( 2 )$$

Where, Δθ is the shift from a direction perpendicular to the object to be measured. By this calculation, the moving speed V of the object to be measured can be obtained. In this case, each of the moving speed Vn for each of the peak wavelengths based on a multi-mode oscillation may be obtained, and then the moving speed Vm of the object to be measured can be finally obtained by the arithmetic average or harmonic average.

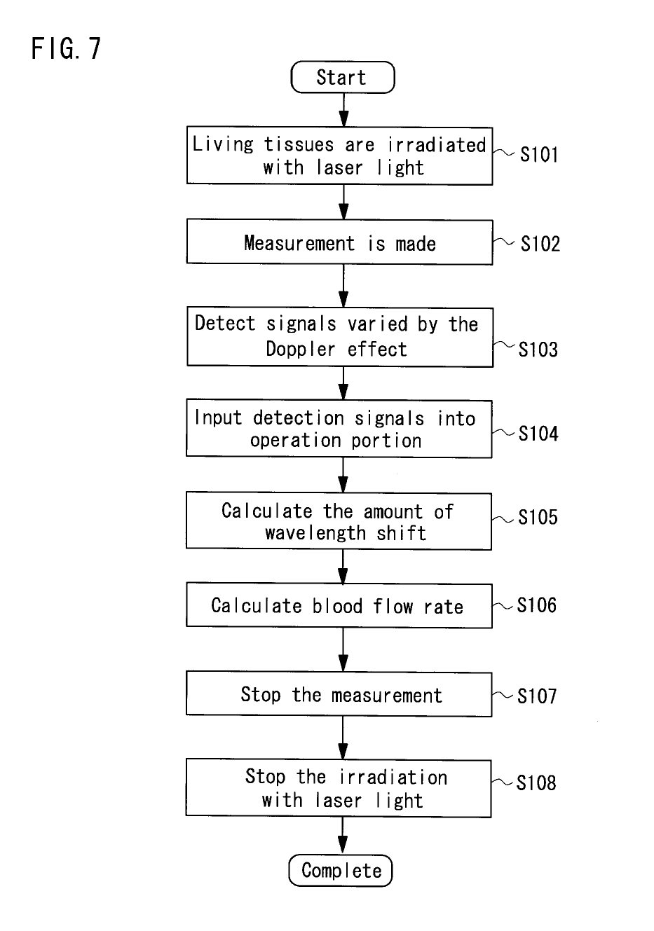

Referring now to a flowchart of FIG. 7, an operation for measuring blood flow rate using an apparatus for measuring blood flow rate will be described. First, the light emitting portion 110 is driven by the driving circuit 120. The light emitting portion 110 irradiates living tissues with multi-mode laser light having plural peak wavelengths (step S101). Along with the irradiation, measurement is made (step S102).

Among the emitted laser light, the light reflected, scattered, or absorbed by blood cells moving in blood vessels experiences wavelength shift or intensity variation caused by the Doppler effect, and the light is then detected by the photo-detector portion 130 (step S103). A detection signal is inputted in the operation portion 140 (step S104). The detection signal may include wavelength data based on spectrum, line width data, intensity data or the like.

The peak wavelength detection portion 142 detects a shifted peak wavelength from the inputted detection signal, and the detected peak wavelength is then provided to the wavelength shift amount calculation portion 144. The wavelength shift amount calculation portion 144 calculates the differences between the detected peak wavelength and the peak wavelength of the irradiated laser light (step S105), and the differences are then provided to the Doppler effect variation amount determination portion 145. The Doppler effect variation amount determination portion 145 determines the amount of variation by the Doppler effect from operation of, for example, the average of differences between each of the peaks, thereby blood flow rate is obtained (step S106).

The processing of the operation will be now described using laser light that has peak wavelengths shown in FIG. 2, as an example. The initial peak wavelength retaining portion 143 may retain the peak wavelengths of the emitted laser light, λP1, λP2, λP3, λP4, and λP5. These wavelengths may be stored in a memory as wavelengths measured when the VCSEL 200 is oscillated at a room temperature. Alternatively, the wavelengths can be obtained from the light reflected from an object being completely at a standstill and irradiated with laser light.

The peak wavelength detection portion 142 analyzes spectrum of the light reflected from the living tissues in order to detect the peak wavelengths whose wavelength is shifted; λP1′, λP2′, λP3′, λP4′, and λP5′. Then, the wavelength shift amount calculation portion 144 calculates differences for each peak; Δλ1=|λP1−λP1′|, Δλ2=|λP2−λP2′|, Δλ3=|λP3−λP3′|, Δλ4=|λP4−λP4′|, and Δλ5=|λP5−λP5′|. The Doppler effect variation amount determination portion 145 calculates the average of Δλ1, Δλ2, Δλ3, Δλ4, and Δλ5, and determines the average as the amount of variation by the Doppler effect.

The blood flow rate calculation portion 146 calculates blood flow rate based on the amount of variation by the Doppler effect. In addition, among the light reflected from the living tissues, the ratio of the light in which wavelength shift (or frequency shift) occurs is proportional to the number of blood cells that follow the movement, and the amount of the shift is proportional to the blood flow rate. In short, there is a relation that the amount of blood flow equals to the number of blood cells multiplied by the blood flow rate. From this relation, the blood flow rate calculation portion 146 can also calculate the amount of blood flow or the number of blood cells.

The method for determining the amount of variation by the Doppler effect is not limited to the use of the arithmetic average of the amount of wavelength shift for each peak; and harmonic average may be alternatively used. In addition, in order to reduce variations in the amount of shift, the maximum and the minimum values of the amount of shift may be excluded from the arithmetic average or harmonic average. In the example, the amount of variation by the Doppler effect is determined from the amount of wavelength shift; however, the amount of variation by the Doppler effect may be determined from the amount of frequency shift for each peak.

As another technique, blood flow rate may be calculated by superimposing and adding two Doppler signals; a Doppler signal obtained from variations over time in intensity of the light reflected from living tissues that are irradiated with laser light, and a Doppler signal obtained from frequency shift in spectrum of the reflected light. This technique utilizes a light absorbing action by the living tissues.

In addition, even if the temperature around the VCSEL changes during the measurement, the spectrum having plural peaks simply varies by a constant amount to a shorter wavelength or a longer wavelength in accordance with the amount of the temperature variation, but the wavelength intervals between each of the oscillation peaks does not change. In other words, there is no influence on determination of the amount of variation by the Doppler effect based on the arithmetic average of the amount of wavelength shift. However, the absolute value of the oscillation peak wavelength varies based on a constant coefficient, and thus it is desirable that the temperature coefficient is previously obtained for data calibration, and is then corrected as appropriate.

In a case where a laser having a single vertical mode property as in a related art is used, operation is performed based on the data of a single oscillation peak, and an error of the data tends to affect results, which lowers accuracy. Furthermore, obtaining the amount of variation by a correct Doppler effect would be difficult if any mode hopping occurs, and thus it is required to provide an extra temperature regulating function in order to accurately control the temperature of the case or ambient temperature. By using a multi-mode VCSEL as a light source, an apparatus for measuring blood flow rate that provides high accuracy and high reliability can be readily obtained without increasing the area of the portion to be measured. Also, the use of a multi-mode VCSEL, which is less expensive, may lead to a significant reduction in fabrication cost.

In the examples described above, a GaAs system VCSEL is illustrated as an example; however, the present invention can also be applicable to a semiconductor laser in which other III-V group compound is used. In the examples described above, a selective oxidation type VCSEL in which a current confining layer is selectively oxidized is illustrated as an example. However, it is not necessarily limited to such examples, and a high resistant area may be formed in a current confining layer by ion implantation or the like. In addition, the oscillation wavelength of the VCSEL may be selected as appropriate in accordance with the object to be measured, such as living tissues or blood vessels.

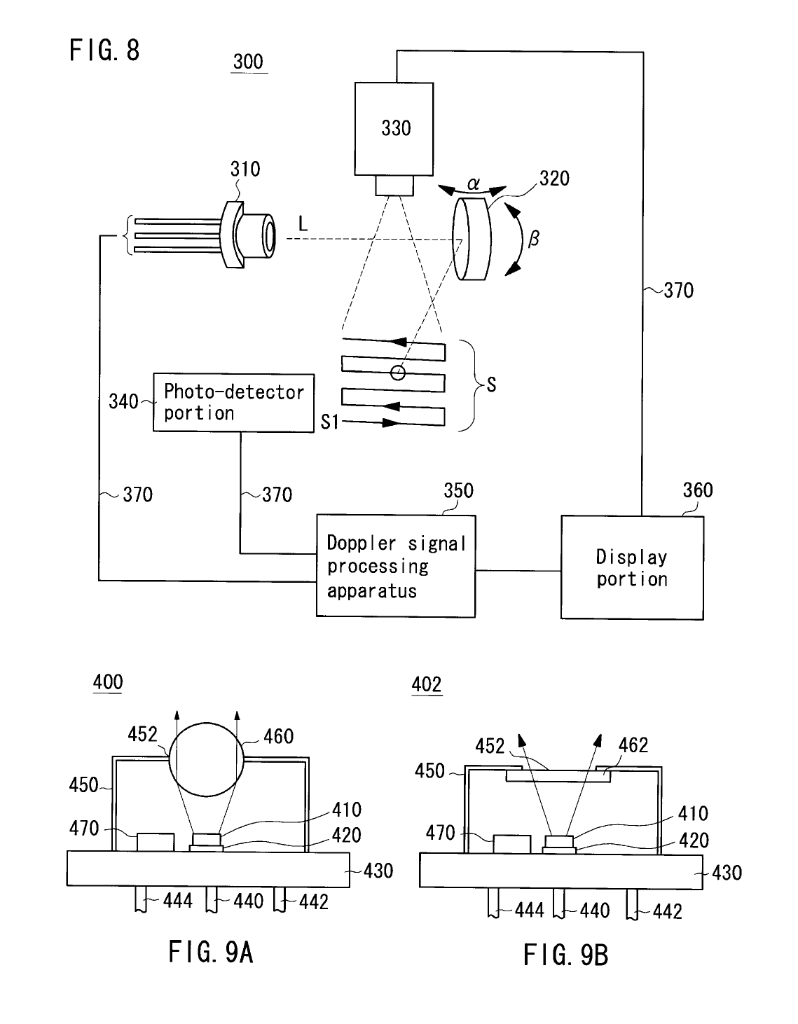

FIG. 8 illustrates an example of a specific configuration of an apparatus for measuring blood flow rate. An apparatus for measuring blood flow rate 300 may include a can type optical module 310 that includes a traverse multi-mode VCSEL therein, a scan mirror 320, being capable of rotating in an a (direction and a β direction that are orthogonal to one another, for forming a scan area S made of a scan line Si on living tissues by reflecting laser light L that is emitted from the optical module 310, an imaging camera 330 for imaging the scan area S, a photo-detector portion 340 for receiving light reflected or scattered from the scan area S, a Doppler signal processing apparatus 350 for generating a driving signal of the VCSEL and performing operation processing of a signal detected from the photo-detector portion 340, a display portion 360 for combining an image imaged by the imaging camera 330 with image data of the blood flow rate and displaying combined data, and signal lines 370 for transmitting a signal between each of the portions.

FIGS. 9A and 9B are examples of a configuration of an optical module shown in FIG. 8. In an optical module 400 shown in FIG. 9A, a chip 410 in which a VCSEL is formed is fixed on a disc-shaped metal stem 430 through a conductive adhesive 420. Conductive leads 440 and 442 are inserted into through holes (not shown) formed in the stem 430. The lead 440 is electrically coupled to an n-side electrode of the VCSEL, and the other lead 442 is electrically coupled to a p-side electrode. A rectangular hollow cap 450 is fixed on the stem 430. In an opening 452 at a center portion of the cap 450, a ball lens 460 is fixed. On the stem 430, a light sensing element 470 for monitoring the light emitting status of the VCSEL is fixed, and an output signal of the light sensing element 470 may be received from the lead 444. In an optical module 402 shown in FIG. 9B, instead of the ball lens in the optical module shown in FIG. 9A, a flat-plate glass 462 is fixed in an opening at a center portion of the cap 450.

The laser light L emitted from the optical module 310 may be scanned by the scan mirror 320 as in the scan line Si in the scan area S on the living tissues. The light reflected from the living tissues during the scanning may be received by the photo-detector portion 340, and a signal detected by the photo-detector portion 340 may be provided through the signal line 370 to the Doppler signal processing apparatus 350. The blood flow rate for the scan line Si or scanning points that make up of the scan line Si may be calculated. The display portion 360 may produce a two-dimensional image from which the distribution of the blood flow rate in the scan area S can be seen, and combine the produced two-dimensional image with image data for the scan area S obtained from the imaging camera 330, and then display a combined image on a monitor. The display portion 360 may provide an image from which a portion of poor blood flow rate (low velocity) or a portion of good blood flow rate (high velocity) can be found at a glance.

The foregoing description of the examples has been provided for the purposes of illustration and description, and it is not intended to limit the scope of the invention. It should be understood that the invention may be implemented by other methods within the scope of the invention that satisfies requirements of a configuration of the present invention.

An apparatus for measuring blood flow rate according to the present invention can, for example, measure the velocity of blood flowing in a living body.

What is claimed is:

1. An apparatus for measuring blood flow rate, comprising:
a light emitting portion for irradiating living tissues with laser light, the spectrum of the laser light having a plurality of peaks;
a photo-detector for detecting at least one of reflection, scattering, or absorption of the laser light; and
an operation portion for calculating blood flow rate based on a difference between the spectrum of the laser light from the light emitting portion and the spectrum of the light detected by the photo-detector.

2. The apparatus for measuring blood flow rate according to claim 1, wherein the light emitting portion comprises a Vertical-Cavity Surface-Emitting Laser diode (VCSEL) device for emitting traverse multi-mode laser oscillation light.

3. The apparatus for measuring blood flow rate according to claim 2, wherein the VCSEL device comprises an active layer and a current confining layer between a first conductivity type semiconductor reflective mirror and a second conductivity type semiconductor reflective mirror, and the current confining layer comprises a high resistant region and a conductive region surrounded by the high resistant region, and the outer diameter of the conductive region is greater than about 5 micrometers.

4. The apparatus for measuring blood flow rate according to claim 1, wherein the operation portion calculates a difference in wavelength shift for each of the peaks, and determines the amount of variation by the Doppler effect based on each of the calculated differences, and calculates blood flow rate from the amount of variation by the Doppler effect.

5. The apparatus for measuring blood flow rate according to claim 4, wherein the operation portion determines the amount of variation by the Doppler effect from the arithmetic average of each of the differences.

6. The apparatus for measuring blood flow rate according to claim 1, wherein the operation portion calculates a difference in frequency shift for each of the peaks, and calculates blood flow rate from the calculated differences.

7. The apparatus for measuring blood flow rate according to claim 1, wherein the operation portion calculates blood flow rate by an addition in which a Doppler signal obtained from variations over time in the intensity of a reflected light and a Doppler signal obtained from frequency shift of the spectrum of the reflected light are superimposed.

8. The apparatus for measuring blood flow rate according to claim 1, wherein the light emitting portion irradiates living tissues with the laser light split in two beams, and wherein the blood flow rate V is calculated using following equation (1), where the frequency of the split laser light beams is f0, the frequencies of light beams being varied by the Doppler effect are f1, f2, the crossing angle of the irradiated laser light is φ, the laser wavelength is λ, and the differential frequency is fd.
$${f}_{d} = \vert {f}_{1} - {f}_{2} \vert = ( {f}_{0} + \frac{V}{λ} sin φ ) - ( {f}_{0} - \frac{V}{λ} sin φ ) \qquad ( 1 )$$
